(19) United States
(12) Patent Application Publication
(10) Pub. No.: US 2026/0158085 A1
(43) Pub. Date: Jun. 11, 2026

(54) ORAL HYGIENE FORMULATION

(71) Applicant: EZ Dent Corp., Sarasota, FL (US)

(72) Inventors: Jonathan Bromboz, Sarasota, FL (US); Thomas Rheinlander, Sarasota, FL (US)

(21) Appl. No.: 19/406,142

(22) Filed: Dec. 2, 2025

Related U.S. Application Data
(60) Provisional application No. 63/728,764, filed on Dec. 6, 2024.

(51) Int. Cl. A61K 35/744; A61K 8/02; A61K 8/19; A61K 8/34; A61K 8/37; A61K 8/44; A61K 8/60; A61K 8/67; A61K 8/73; A61K 8/92; A61K 8/9789; A61K 8/99; A61K 31/047; A61K 31/122; A61K 31/198; A61K 31/23; A61K 31/593; A61K 33/10; A61K 36/889; A61Q 11/00

(52) U.S. Cl. CPC A61K 35/744; A61K 8/0225; A61K 8/19; A61K 8/345; A61K 8/375; A61K 8/44; A61K 8/602; A61K 8/67; A61K 8/737; A61K 8/922; A61K 8/9789; A61K 8/99; A61K 31/047; A61K 31/122; A61K 31/198; A61K 31/23; A61K 31/593; A61K 33/10; A61K 36/889; A61Q 11/00; A61K 2800/48; A61K 2800/92

(57) ABSTRACT

An oral hygiene powder formulation and a method of administering a therapeutic consumable preparation are disclosed. The oral hygiene powder formulation includes coconut oil or MCT powder, remineralizing agent(s), vitamins, supplement(s), probiotic(s), sugar alcohol, optionally together with emulsifiers and/or thickeners and/or stabilizers and/or sweeteners and/or flavorants. The probiotic includes one or more strains of Streptococcus Salivarius.

### CROSS-REFERENCE TO RELATED APPLICATIONS

This U.S. patent application claims priority under 35 U.S.C. § 119(e) to U.S. Provisional Application No. 63/728,764 filed on Dec. 6, 2024, the contents of which are hereby incorporated by reference in its entirety.

### TECHNICAL FIELD

The present disclosure is directed to an oral hygiene formulation and method of administering an oral hygiene formulation, e.g., for treating dry mouth, cavities, and/or gum disease.

### BACKGROUND

There is a condition that patients suffer from called Dry Mouth Syndrome. People with dry mouth suffer from reduced production, flow and quality of saliva from their saliva glands. This leads to difficulty with speaking, eating, swallowing, and uncomfortable sensations in the mouth such as tingling, soreness, aching or parched feeling. Additionally, Dry Mouth Syndrome may lead to rampant, recurring dental cavities and gum disease.

Dental decay (cavities) and gum disease are caused by infectious, invasive types of bacteria. These disease-causing bacteria, festering in the mouth, burrow into blood vessels and travel to other parts of the body to lodge in major organs, contributing to the leading fatal diseases such as heart attack, stroke, and cancer.

Conventional delivery systems to help fight dental decay such as tablets, lozenges, mouthwashes, chewing gum, and tooth pastes only deliver therapeutic agents (e.g., sugar alcohol) over a brief period of time, which reduces the effectiveness of the therapeutic agent as compared to delivery over a prolonged period of time (e.g., over the course of a day). Additionally, mouth washes and tooth pastes may be toxic if consumed or otherwise contain toxic ingredients.

Gum disease causes generalized inflammation and leads to significant immune system problems in many areas of the human body. Immune system dysfunction and inflammation are a central element of a large percentage of human diseases and conditions.

Gum disease may cause inflammation that leads to life threatening conditions. Inflammation is beginning to be recognized in the medical community as a cause of cardiovascular disease which leads to heart attacks, stroke, autoimmune disease and thromboembolism.

Current techniques for treating gum disease including blasting the bad oral bacteria with powerful antibiotics, lasers, surgery, antibacterials or disinfectants. However, these treatments have failed to demonstrate long-term success.

There is a need to develop a treatment and/or product that fights the symptoms and effectively treats or prevents pathogenic bacteria that cause oral diseases, cavities and gum disease.

According to the disclosure, there is provided an oral hygiene formulation and method of administering an oral hygiene formulation used to prevent pathogenic bacteria from causing cavities and gum disease as well as treat Dry Mouth Syndrome, as set forth in the appended claims.

### SUMMARY

An object of the disclosure is to provide an oral hygiene formulation for the treatment and/or prevention of pathogenic bacteria from causing cavities and gum disease.

In one aspect, the oral hygiene formulation includes one or more probiotics, nutrient(s) (e.g., a remineralizer and/or a supplement), a sugar alcohol, coconut oil (medium chain triglycerides (MCT)), vitamin(s), thickeners and/or carriers and/or flavorants combined as a powder mixture that may be added to a liquid (e.g., water) for administering to a patient (person) for treating oral diseases.

The method of administration includes a therapeutic powder of the oral hygiene formulation that is self-administered by being mixed into a liquid (e.g., water). The mixture is then sipped, swished around the mouth and swallowed to maintain better oral health and to fight pathogenic bacteria that cause cavities, gum disease, and other oral problems such as dry mouth. The therapeutic benefits may be achieved through daily administration of the oral hygiene powder, merely as an example.

Pursuant to an implementation, the oral hygiene powder formulation includes therapeutic and non-therapeutic ingredients. The therapeutic powder ingredients medium chain triglycerides (MCT), a sugar alcohol (e.g., xylitol), a remineralizing agent (e.g., arginine and/or calcium (e.g., calcium carbonate)), and a probiotic (e.g., Streptococcus Salivarius K12 and/or Streptococcus Salivarius M18), optionally including a vitamin (e.g., vitamin K and/or vitamin D). The non-therapeutic powder ingredients include a thickener (e.g., a gum thickener such as guar gum) and a carrier (e.g., trehalose), optionally including a stabilizer (e.g., sea salt), a sweetener (e.g., sucralose), and a flavorant (e.g., cocoa or plain powder).

The oral hygiene powder formulation pursuant to the disclosure may have a balanced pH, such that when mixed with liquid (e.g., water), the pH of the mixture remains balanced and neutral (e.g., about 7.2 pH) when consumed. A balanced pH may be beneficial for many reasons, including enamel protection and reducing or preventing demineralization, facilitating salivary function, and helping fight or inhibit pathogenic bacteria (e.g., Streptococcus mutans and Lactobacillus spp.) that thrive in acidic conditions.

Additional features and advantages of oral hygiene formulation described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein and together with the description serve to explain the principles and operations of the claimed subject matter.

### DETAILED DESCRIPTION OF THE EMBODIMENTS

In one aspect, there is disclosed herein an oral hygiene formulation that may be designed as a premix powder composition added to a liquid (e.g., water). The oral hygiene formulation includes therapeutic ingredients and non-therapeutic ingredients combined in a single pack that is added and mixed with the liquid. The therapeutic ingredients comprise one or more strains of probiotics, nutrient(s) (e.g., a remineralizer and/or a supplement), a sugar alcohol, coconut oil or MCT powder, and vitamin(s). Non-therapeutic ingredients may be included such as thickeners and/or flavorants and/or carriers and/or sweeteners and/or dispersants to facilitate easy and convenient self-administration.

According to an aspect, the oral hygiene formulation is made into a powder that is sealed in container such as a package, pouch, a sealed bottle cap, or packet. The probiotic strain(s) used herein promote dental health (e.g., gum health) and cavity prevention, and need to be kept dry and water free until being dispensed. If moisture reaches the probiotics, the water will activate the germination of the probiotic bacteria.

The therapeutic oral hygiene formulation, self administered as a beverage (e.g., a powder formulation mixed into a liquid such as water), includes a combination of multiple overlapping, co-functional ingredients that provide synergistic benefits to fight tooth decay, cavities, gum disease, and dry mouth.

The invention is a method and powder formulation for self administering a composition of therapeutically effective amounts of coconut oil (e.g., medium chain triglycerides), sugar alcohol(s) (e.g., xylitol), one or more vitamins (e.g., vitamin K and/or vitamin D), a remineralizer (e.g., arginine), a one or more supplements (e.g., calcium carbonate), and a probiotic of one or more isolated strains of probiotic bacteria (e.g., Streptococcus salivarius) that inhibit the bacteria that cause dental disease. In this method of administration the key to the more consumer friendly and all day effectiveness and symptom relief is the ability to self administer from a convenient bottle (e.g., water bottle or cup) by mixing the oral hygiene powder formulation in water or other liquid. A single dose of the disclosed oral hygiene formulation may be mixed into 6 oz to 16 oz of water, merely as an example. The mixture of oral hygiene powder formulation and liquid may have the texture of a smoothie, helping with oral lubrication and an overall pleasant taste and feel.

Utilization of coconut oil, which may be in the form of medium chain triglyceride (MCT) powder, is an essential part of administration. Coconut oil or MCT powder increases the bioavailability of nutrients, vitamins, and probiotics. The coconut oil creates a silky, moistening, smooth coating inside the mouth when mixed and ingested during self-administration, providing benefits for dry mouth. The MCT powder, emulsified into water, coats the teeth and tongue and helps prevent the attachment of pathogenic bacteria and acts as a soothing and moistening lubricant and emollient to the inside of the mouth, gums, and throat providing relied from the dry mouth symptoms. Coconut oil or MCT powder is anti-bacterial, anti-viral, anti-fungal, and anti-candida, providing benefits for teeth and gums, such as by helping suppress bacteria that causes tooth decay and cavities. Additionally, coconut oil or MCT powder interacts with oil soluble vitamins, supplements, nutrients, chemicals and compounds to form complexes that are more easily transportable across membranes, producing heightened assimilation of compounds into the body. For a beneficial therapeutic advantage, the concentration of coconut oil or MCT powder should amount to 30 wt % to 70 wt % of the total weight of the formulation, particularly 40 wt % to 60 wt %, and more particularly between 45 wt % and 50 wt %. The synergistic effect of combining greater amounts of coconut oil or MCT powder, than is normally used, in beverages with vitamins, supplements, and nutrients is a synergistically increased absorption of the above ingredients and transport into the cells and organs of the body with increased availability. The method of administration in a beverage described allows higher therapeutic levels of coconut oil to be able to be consumed as a pleasant beverage without unpleasantness or upsetting the digestion.

The sugar alcohol (e.g., xylitol) have the therapeutic effect of inhibiting the growth of cavity-causing bacteria leading to a decrease in tooth decay. Pursuant to an implementation, the oral hygiene formulation uses xylitol, a non-sugar sweetener that comes from birch trees, peaches, and other plants, which functions to suppress the growth of bad bacteria in the mouth that causes cavities as well as enhance taste/sweetness during administration. Xylitol has also been shown to work synergistically with the probiotic(s) to suppress pathogenic bacteria in the mouth and gums. Additionally, xylitol binds to pathogenic bacteria and stalls their metabolism, causing the bacteria to degenerate. Importantly, xylitol is much more effective if delivered over a long period of time, as is the case with the disclosed formulation and method of consumption, in contrast to existing solutions employing xylitol (e.g., chewing gum or mouth washes). A therapeutically effective amount of sugar alcohol (e.g., xylitol) described herein ranges from 5 wt % to 15 wt %, particularly 7 wt % to 13 wt %, more particularly 10 wt % to 11 wt %.

Therapeutic vitamins, such as Vitamin D3 (e.g., cholecalciferol) and Vitamin K2 (e.g., menaquinone), include therapeutic benefits to support bone health, stronger tooth enamel, and healthier calcium levels. The vitamin D3 and K2 may be contained in a combined amount of 0.2 wt % to 1.3 wt %. Pursuant to an implementation, a therapeutically effective amount of vitamin D3 ranges from 0.2 wt % to 1.2 wt %, particularly 0.5 wt % to 0.9 wt %, and a therapeutically effective amount of vitamin K2 ranges from 0.0001 wt % to 0.0003 wt %, particularly about 0.0002 wt %.

A therapeutic remineralizer, such as arginine (e.g., L-Arginine), is an amino that helps rebuild and strengthen weak tooth enamel and re-mineralize the teeth. Arginine also acts as a pH-elevating agent to help with sensitivity and suppressing cavity-causing bacteria. Therapeutically effective amounts of arginine described herein range from 0.2 wt % to 1.2 wt %, particularly 0.5 wt % to 0.9 wt %.

A therapeutic supplement or further remineralizing agent, such as calcium (e.g., calcium carbonate (CaCO₃), calcium citrate (Ca₃(C₆H₅O₇)₂), calcium phosphate (Ca₃(PO₄)₂), may be added to promote calcium supplementation in an amount of from 0.2 wt % to 1.2 wt %, particularly 0.5 wt % to 0.9 wt %.

The combination of arginine with calcium in the disclosed proportions improves the remineralization properties and helps repair tooth enamel. The arginine and calcium may together provide a remineralizing agent in an amount of 0.2 wt % to 2.5 wt %.

Non-therapeutic ingredients including thickener, carrier, and dispersant may be added to facilitate delivery of the therapeutic components. Pursuant to an implementation, the thickener (e.g., guar gum or xanthan gum) is provided in an amount of 2 wt % to 8 wt %, particularly from 4 wt % to 6 wt %, to improve texture and/or emulsification when the powder is mixed into the liquid, as well as help with mouth lubrication and soothing sore mouth tissues. Pursuant to an implementation, a carrier/dispersant (e.g., trehalose) may be provided in an amount of 7 wt % to 27 wt %, particularly 15 wt % to 20 wt %, to facilitate homogenous mixture and/or prevent clumping).

Further non-therapeutic ingredients such as sweetener, flavorant, and stabilizer may be added for taste masking and product longevity or shelf-life. Pursuant to an example, a sweetener such as sucralose or stevia is provided in an amount of 0.1 wt % to 1 wt %, particularly 0.3 wt % to 0.8 wt %. Natural flavors such as those derived from fruits and vegetables may be provided in an amount of 0.3 wt % to 1 wt %. A flavorant such as cocoa powder or plain powder may be provided for flavor in an amount of 7 wt % to 27 wt %, particularly 15 wt % to 20 wt %. Salt (e.g., sea salt) may be provided as a stabilizer and/or to remove moisture in the packet (sealed packaging) to help preserve the probiotic in an amount of 0.4 wt % to 2.4 wt %, particularly 1 wt % to 2 wt %.

Therapeutic probiotics including Streptococcus Salivarius (S. Salivarius) inhibits the growth of pathogenic bacteria in the mouth by producing bacteriocins that synergistically reduce the number of pathogenic bacteria causing cavities and gum disease. The probiotic bacteria used herein has been shown to flourish and replace the pathogens, excluding growth of other bacteria in the mouth. Further, S. salivarius promotes heart health and reduce heart disease, as bad (pathogenic) bacteria in the gums can lead to poor heart health, inflammation, and possibly cause heart disease. S. salivarius is an oral microbe that secretes a bacteriocin-like inhibitory substance (BLIS), a type of antimicrobial peptide that inhibits the growth of other bacteria. There is a synergistic enhancement between this probiotic and the sugar alcohol (e.g., xylitol) to help prevent cavities and gum disease. Pursuant to an implementation, the S. salivarius ingredient may be S. sSalivarius K12 or S. salivarius M18, or a blend of S. salivarius K12 and M18. S. salivarius K12 and M18 exhibit broad-spectrum bacteriocin effects. The specification of S. salivarius in K12 or M18 ingredient is approximately one hundred billion colony-forming units (cfu) per gram. The probiotic may be produced by fermenting S. salivarius K 12 or M18 in an enzymatically-digested milk-based growth media, where the cels after growth are removed and may be blended with stabilizing agents (e.g., non-cariogenic sugars) and milled into powder form. Therapeutically effective amounts of probiotic(s) described herein range from 0.04 wt % to 0.14 wt %, particularly 0.8 wt % to 0.1 wt %.

Without wishing to be bound by theory, S. salivarius K12 helps inhibit oral pathogens through a mechanism that produces bacteriocins (e.g., salivaricin A2 and B) that inhibit a range of oral pathogens to target bad breadth and support upper respiratory health, while S. salivarius M18 helps inhibit oral pathogens through a mechanism that produces different bacteriocins (e.g., salivaricin A2, MPS, and salivaricin M) that are effective against bacteria that cause plaque formation and cavities (e.g., S. mutans and Actinomyces viscosus).

Other probiotics, additionally or alternatively, are also contemplated. For example, Lactobacillus reuteri and/or Lactobacillus rhamnosus may be included alone, in combination, or as part of a probiotic blend with S. salivarius. Lactobacillus reuteri may help combat Streptococcus mutans, a cause of tooth decay, and Lactobacillus rhamnosus may help prevent cavities.

### Example

In one aspect, the oral hygiene formulation may comprise a premix powder totaling 22000 mg, and may include:

| | | | | PERCENT |
|---|---|---|---|---|
| INGREDIENT | AMOUNT | | PERCENTAGE | RANGE |
| Medium Chain | 9894.3 | mg | 45 | PLUS- |
| Triglycerides | | | | MINUS |
| (MCT) Powder | | | | 15% |
| Xylitol | 2283.3 | mg | 10.4 | PLUS- |
| | | | | MINUS |
| | | | | 5% |
| Calcium | 152.22 | mg | 0.7 | PLUS- |
| Carbonate | | | | MINUS |
| | | | | 0.5% |
| L-Arginine | 152.22 | mg | 0.7 | PLUS- |
| | | | | MINUS |
| | | | | 0.5% |
| Sea Salt | 304.44 | mg | 1.4 | PLUS- |
| | | | | MINUS |
| | | | | 1% |
| Trehalose | 3805.5 | mg | 17.3 | PLUS- |
| (TREHA ®) | | | | MINUS |
| | | | | 10% |
| Vitamin D3 | 152.22 | mg | 0.7 | PLUS- |
| (100,000 IU/g) | | | | MINUS |
| | | | | 0.5% |
| Vitamin K2 | 45.6 | mcg | 0.0002 | PLUS- |
| (MK-7 1% | | | | MINUS |
| PWD) | | | | 0.0001% |
| Cocoa Powder | 3805.5 | mg | 17.3 | PLUS- |
| | | | | MINUS |
| | | | | 10% |
| Guar Gum | 1141.65 | mg | 5.2 | PLUS- |
| | | | | MINUS |
| | | | | 3% |
| Natural Flavor | 190.275 | mg | 0.86 | PLUS- |
| | | | | MINUS |
| | | | | 0.5% |
| Sucralose | 114.165 | mg | 0.52 | PLUS- |
| | | | | MINUS |
| | | | | 0.4% |
| Probiotic | 20 | mg | 0.09 | PLUS- |
| | | | | MINUS |
| | | | | 0.05% |

As mentioned above, the powder formulation is based on a total of 22000 mg merely as an example. The weight percentages (wt %) are based on a total weight of the powder composition.

As specified above, the oral hygiene powder formulation includes therapeutically effective amounts of coconut oil (e.g., MCT powder), sugar alcohol (e.g., xylitol), one or more vitamins (e.g., vitamin K and/or vitamin D), one or more remineralizers or supplements (e.g., arginine and/or calcium carbonate), and a probiotic of one or more isolated strains of probiotic bacteria (e.g., S. salivarius, in particular S. salivarius K12 and/or M18) together with non-therapeutic agents such as a gum thickener (e.g., guar gum or xanthan gum) and/or carriers (e.g., trehalose) and/or stabilizers (e.g., sea salt), and/or flavorant (e.g., cocoa powder or plain powder) and/or sweeteners (e.g., sucralose or stevia), and/or natural flavors, which is provided in a powder composition that is mixed with a liquid (water) for self-administration. The powder composition may be pre-packaged, for example in a sealed container such as a pouch, packet, or bottle cap with removable foil.

The probiotic may include Streptococcus Salivarius, particularly S. salivarius BLIS K12 and/or S. salivarius BLIS M18. The probiotic strain may be manufactured by BLIS Probiotics (BLIS Technologies Ltd), Dunedin, New Zealand. The specification of S. salivarius in BLIS K12 or M18 ingredient is one hundred billion (1011) colony-forming units (cfu) per gram. The S. salivarius may be fermented in an enzymatically-digested milk-based growth media. The cells after growth are removed, blended with stabilizing agents that mainly consist of sugars (non cariogenic), then freeze-dried. When exposed to moisture, the bacteria are rejuvenated from a dormant state.

The coconut oil may be provided in the form of MCT powder (e.g., MCT powder 50%), which helps mixing with water. The MCT powder acts as a therapeutic agent by providing anti-bacterial, anti-viral, anti-fungal, and anti-candida benefits, acts as a carrier that facilitates a smooth, silky feel/taste, and acts as a delivery system for the administration of other therapeutic ingredients in the formulation.

Calcium carbonate together with arginine help protect, remineralize, and rebuild tooth structure.

Sugar alcohol (e.g., xylitol) helps suppress growth of cavity causing bacteria.

The natural flavor may be derived from fruits, herbs, vegetables, barks, or roots, as is known to enhance taste.

The formulation is intended to be self administered one or more times a day, preferably over a prolonged period of the day. Whenever the consumer feels they need to, they just take a small sip, do a slow swish, and swallow. “Sip, Swish, Swallow” throughout the day, when they want to get relief or just want to take a drink because it taste good. The powder formulation has a balanced pH (e.g., about 7.2 pH) that mirrors the natural oral environment, reinforcing enamel integrity, supporting salivary defense systems, reducing pathogenic bacterial activity, and maintaining mucosal comfort. This balance makes such formulations therapeutically advantageous for both caries prevention and overall oral hygiene maintenance.

Thus, the ingredients are delivered over a much greater amount of time and the therapeutic dose is renewed over and throughout the day, markedly increasing the effectiveness of the therapeutic ingredients.

The therapeutic ingredients sugar alcohol (xylitol), oil (coconut oil, MCT), remineralizer (arginine), vitamins (D & K), supplement (calcium carbonate), and probiotic(s) (S. salivarius such as S. salivarius K12 or M18) work synergistically to fight cavities and gum disease, which may also lead to cardiovascular benefits by reducing the risks associated with heart disease. The non-therapeutic ingredients including thickeners or emulsifier (e.g., guar gum) and/or carriers (e.g., trehalose) and/or stabilizers (e.g., sea salt), and/or flavorant (e.g., cocoa or plain powder) and/or sweeteners (e.g., sucralose or stevia), and/or natural flavors promote product stability, texture or thickness, longevity, and taste once mixed in with the liquid together with the therapeutic ingredients.

The therapeutic ingredients of MCT powder, sugar alcohol(s), and probiotics potentiate each other's effects and produce powder synergistic anti-dental disease therapeutic effects. Self-administering the oral hygiene formulation provides benefits including: cavities are decreased, gum disease is decreased, quality of life is improved, tooth loss is decreased, tooth retention is increased, morbidity is decreased, susceptibility to life threatening disease is decreased, longevity is increased, and overall health is increased.

Because of lack of lubrication in the mouth people with dry mouth have difficulty eating and swallowing. This method of administration of lubricants and moisteners as disclosed herein through self-administration in a beverage (e.g., by pouring the premix powder into water and mixing) markedly enhances chewing, swallowing, and taking pills, vitamins, and medications in dry mouth suffers.

Essential ingredients include coconut oil or MCT powder, remineralizing agent(s), supplement(s), probiotic(s), and sugar alcohol, optionally together with vitamins, emulsifiers and/or thickeners and/or stabilizers and/or sweeteners and/or flavorants. The ingredients are each provided in a powder form and combined into a mixture. The powder mixture of the disclosed oral hygiene formulation may be dosed into individual pre-mixtures (e.g., a single dose of the ingredients (including proportions) in a sealed container). This approach provides advantages associated with accurate dosage of various the therapeutic agents to improve efficacy. Alternatively, the powder mixture of the disclosed oral hygiene formulation may be packaged in multi-dose form, e.g., a bulk powder in a sealed container that can be scooped into the liquid (e.g., water) for mixing with the liquid and consumption/use by the individual.

Unlike the majority of beverages, the disclosed oral hygiene powder formulation when mixed with water provides a convenient method of delivery that buffers an alkaline pH to help counteract acidity in the mouth, which can produce an irritating feeling in the mouth especially in people suffering from dry mouth. The disclosed method motivates self administration delivery of therapeutic ingredients over prolonged time. Additionally, the oral hygiene powder formulation is free from harmful chemicals (unlike mouth wash, for example), enabling its consumption and therapeutic benefits of its active (therapeutic) ingredients).

Even though the present disclosure has been described in detail with reference to specific examples, it will be appreciated that the various modifications and changes can be made to these examples without departing from the scope of the present disclosure as set forth in the claims. For example, minerals (e.g., fluoride and/or phosphorous), herbals (e.g., green tea, ginger, clove), plant materials (e.g., mint), vitamins (e.g., vitamin C, vitamin B), oils (e.g., olive oil) may be added to the oral hygiene formulation for enhanced therapeutic effects. In certain circumstances, ingredients may be substituted for less therapeutically effective ingredients, such as utilizing erythritol or sorbitol as the sugar alcohol, without departing from the scope of the disclosure. It is anticipated and intended that future developments will occur in the technologies discussed herein, and that the disclosed product, composition and/or method will be incorporated into such future developments. Thus, the specification and the drawings are to be regarded as an illustrative thought instead of merely restrictive thought.

It should be understood that references to a single element are not necessarily so limited and may include one or more of such element. Further, all numbers expressing dimensions, ratios and the like, used in the specification and claims, are to be understood to encompass tolerances and other deviations (e.g., within +/−1%) as represented by the term “about” or “approximately.”

The use of “e.g.” in the specification is to be construed broadly and is used to provide non-limiting examples of embodiments of the disclosure, and the disclosure is not limited to such examples. Uses of “and” and “or” are to be construed broadly (e.g., to be treated as “and/or”). For example and without limitation, uses of “and” do not necessarily require all elements or features listed, and uses of “or” are inclusive unless such a construction would be illogical.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary. Further, the use of “at least one of” is intended to be inclusive, analogous to the term and/or. As an example, the phrase “at least one of A, B and C” includes A only, B only, C only, or any combination thereof (e.g. AB, AC, BC or ABC). Additionally, use of adjectives such as first, second, etc. should be read to be interchangeable unless a claim recites an explicit limitation to the contrary.

What is claimed is:

1. An oral hygiene powder formulation, comprising:
medium chain triglycerides or coconut oil;
a sugar alcohol;
a remineralizing agent; and
a probiotic.

2. The oral hygiene powder formulation of claim 1, wherein the sugar alcohol comprises xylitol.

3. The oral hygiene powder formulation of claim 1, wherein the remineralizing agent comprises arginine and/or calcium.

4. The oral hygiene powder formulation of claim 3, wherein the calcium is calcium carbonate.

5. The oral hygiene powder formulation of claim 1, wherein the probiotic comprises Streptococcus Salivarius K12 and/or Streptococcus Salivarius M18.

6. The oral hygiene powder formulation of claim 1, further comprising thickeners and/or emulsifiers and/or sweeteners.

7. The oral hygiene powder formulation of claim 6, wherein the thickeners and/or the emulsifiers and/or the sweeteners include cocoa, trehalose, and guar gum.

8. The oral hygiene powder formulation of claim 1, further comprising vitamin K and/or vitamin D.

9. The oral hygiene powder formulation of claim 1, wherein the medium chain triglycerides or coconut oil amounts to 30 wt % to 70 wt %, the sugar alcohol amounts to 5 wt % to 15 wt %, the remineralizing agent amounts to 0.2 wt % to 2.5 wt %, and the probiotic amounts to 0.04 wt % to 0.14 wt %.

10. A method of treating dry mouth, comprising administering the oral hygiene powder formulation of claim 1 by mixing with water.

11. A method of treating dental cavities and/or gum disease, comprising administering the oral hygiene powder formulation of claim 1 by mixing with water.

12. An oral hygiene powder formulation, comprising:
therapeutic ingredients and non-therapeutic ingredients;
the therapeutic ingredients including medium chain triglycerides (MCT), a sugar alcohol, arginine, calcium, and a probiotic;
the non-therapeutic ingredients including a gum thickener and a carrier.

13. The oral hygiene powder formulation of claim 12, wherein the gum thickener includes guar gum and the carrier includes trehalose.

14. The oral hygiene powder formulation of claim 12, wherein the non-therapeutic ingredients further include one or more of cocoa, sucralose, and sea salt.

15. The oral hygiene powder formulation of claim 14, wherein the cocoa amounts to 7 wt % to 27 wt %, the sucralose amounts to 0.1 wt % to 1 wt %, and the sea salt amounts to 0.4 wt % to 2.4 wt %.

16. The oral hygiene powder formulation of claim 12, wherein the MCT amounts to 30 wt % to 70 wt %, the sugar alcohol amounts to 5 wt % to 15 wt %, the arginine amounts to 0.2 wt % to 1.2 wt %, the calcium amounts to 0.2 wt % to 1.2 wt %, and the probiotic amounts to 0.04 wt % to 0.14 wt %.

17. The oral hygiene powder formulation of claim 12, wherein the gum thickener amounts to 2 wt % to 8 wt % and the carrier amounts to 7 wt % to 27 wt %.

18. The oral hygiene powder formulation of claim 12, wherein the probiotic is Streptococcus Salivarius.

19. The oral hygiene powder formulation of claim 12, wherein the therapeutic ingredients further include a vitamin including vitamin D and/or vitamin K, and wherein the vitamin amounts to 0.2 wt % to 1.3 wt %.

20. A premix packet, comprising the oral hygiene powder formulation of claim 12.
